(19) United States
(12) Patent Application Publication
(10) Pub. No.: US 2013/0338086 A1
(43) Pub. Date: Dec. 19, 2013

(54) METHODS FOR PURIFYING MONOSACCHARIDE MIXTURES CONTAINING IONIC IMPURITIES

(71) Applicant: Aptalis Pharma Canada Inc., (US)

(72) Inventors: Edward Delaney, Princeton, NJ (US); Anil Oroskar, Oak Brook, IL (US)

(73) Assignee: Aptalis Pharma Canada Inc., Mont-Saint-Hilaire (CA)

(21) Appl. No.: 13/735,805

(22) Filed: Jan. 7, 2013

Related U.S. Application Data
(63) Continuation of application No. 13/481,447, filed on May 25, 2012, now abandoned.
Application No. 13/481,447 is a continuation of application No. PCT/US2010/059244, filed on Dec. 7, 2010.
(60) Provisional application No. 61/267,127, filed on Dec. 7, 2009.

(51) Int. Cl. C07H 1/06; C07H 3/02

(52) U.S. Cl. CPC C07H 1/06; C07H 3/02
USPC 514/23; 536/127; 536/1.11

(57) ABSTRACT

Disclosed herein are methods for separating ionic impurities from monosaccharide processing streams using simulated moving bed chromatography.

This application claims the benefit of U.S. Provisional Application No. 61/267,127, filed Dec. 7, 2009, which is hereby incorporated by reference.

### BACKGROUND

A variety of methods exist for separating polar organic substances from ionic substances. Many of these methods require multiple purification steps and do not achieve complete separation. For example, U.S. Pat. Nos. 5,968,362 and 6,391,204 describe methods involving the use of an anionic exchange resin to remove heavy metals and acid from organic substances. However, these methods are not amenable to complete acid removal, nor do they allow for removal of inorganic and organic cations and anions simultaneously. Similarly, U.S. Pat. Nos. 5,538,637 and 5,547,817 describe methods for separating acids from sugar molecules. However, these methods are limited to separating acids and are not applied to the simultaneous removal of all forms of inorganic and organic cations and anions. Additionally, U.S. Patent Publication Nos. 2009/00556707 and 2008/0041366 disclose using an ion exchange resin for separating first calcium sulfate then acids from sugar mixtures. However, these processes require regeneration of the resin and thus are not amenable to a continuous process.

Accordingly, a need exists for improved methods for separating ionic substances, including inorganic and organic ions, from organic substances, which is preferably efficient and more preferably compatible with a continuous industrial process. These needs and other needs are addressed through the use of the disclosed processes.

### SUMMARY

The present inventors have discovered that ionic impurities can be removed from a monosaccharide starting material in a continuous process using simulated moving bed chromatography. Unlike other purification techniques, the process does not need to be stopped to regenerate a resin, nor do multiple different purification steps need to be performed. This process provides improved speed at reduced cost.

The present invention relates to methods for continuously and simultaneously separating both inorganic and organic ionic impurities from a monosaccharide-containing process stream. The invention also relates to methods of separating an ionic impurity from a saccharide containing process stream.

The invention also relates to L-glucose substantially free (e.g., containing less than 5, 4, 3, 2, 1, 0.5, 0.3, 0.2, 0.1% by weight, based on 100% total weight of the L-glucose, including its impurities) or completely free of ionic (e.g., cationic and/or anionic organic and/or inorganic) impurities. Preferably, the L-glucose is also substantially pure, i.e., is 95, 96, 97, 98, 99, 99.5, 99.7, 99.8, or 99.9% pure (by weight), based on the total weight of the L-glucose, including its impurities). For instance, the L-glucose can be prepared by the simulated moving bed chromatography process of the present invention. In one embodiment, the L-glucose is free or substantially free of all, or one, two, three, or four or more of the following ionic impurities:

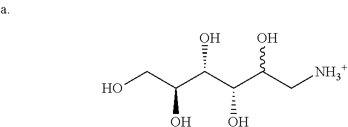 [SMILES: [NH3+]CC(O)[C@H](O)[C@@H](O)[C@@H](O)CO]

b. (3S,4S,5S)-2,3,4,5,6-pentahydroxyhexan-1-aminium
c. CH₃NH₃⁺ (methanaminium)
d. Na⁺ (sodium),
e. NH₄⁺ (ammonium); and
f. SO₄²⁻ (sulfate).
   In another embodiment, the L-glucose is free or substantially free of all, or one, two, three, or four or more of the following ionic impurities:
a. The monosodium salt of

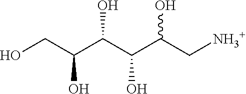 [SMILES: [NH3+]CC(O)[C@H](O)[C@@H](O)[C@@H](O)CO]

b. (3S,4S,5S)-2,3,4,5,6-pentahydroxyhexan-1-aminium
c. CH₃NH₃⁺ (mcthanaminium)
d. Na₂SO₄
e. (NH₄)₂SO₄; and
f. H₂Mo₇O₂₄⁻⁴.
   All of these impurities may be formed during preparation of the L-glucose bulk material. The L-glucose preferably has a conductivity of less than about 750, less than about 500, less than about 300, less than about 250, less than about 200, less than about 150, less than about 100, less than about 50, or less than about 10 μSiemens/cm.

Yet another embodiment is a pharmaceutical composition comprising the L-glucose of the present invention (e.g., that made by the process of the present invention) and a pharmaceutically acceptable carrier or diluent.

Yet another embodiment is a method for colonic cleansing by administering to a subject (e.g., a human) an effective amount of the L-glucose of the present invention (e.g., that made by the process of the present invention).

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURE

The accompanying FIGURE, which is incorporated in and constitutes a part of this specification, illustrates several aspects described below.

FIG. 1 is an illustration of a simulated moving bed chromatography.

### DETAILED DESCRIPTION

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more of the disclosed compounds, the disclosed compounds in combination with other pharmaceutically active compounds, or the disclosed compounds, solvates or diluents of the compounds as defined herein with other pharmaceutically acceptable ingredients.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

A weight percent (wt. %) of a component, unless specifically stated to the contrary, is based on the total weight of the formulation or composition in which the component is included.

By “feed” is meant a chemical process stream to be separated.

By “sorbent” is meant a material, such as a semi-stationary material, that interacts with the feed, and allows slower or faster movement of substances in the feed to be separated.

By “desorbant” is meant a liquid that is added to effect the separation.

By “extract” is meant an exit stream containing slower moving component(s) being separated.

By “raffinate” is meant an exit stream containing the faster-moving component(s) being separated.

By “eluted” or “eluting” is meant the process of passing (either actively or passively) an eluent through a chromatography resin.

“Dianion” is used herein to generally refer to any ionic species having a −2 formal charge.

The term “monosaccharide,” as used herein, can include any monosaccharide, such as, for example, mannose, glucose (dextrose), fructose (levulose), galactose, xylose, ribose or any combination of any of the foregoing. In a preferred embodiment, the monosaccharide is L-glucose. In another preferred embodiment, the monosaccharide is a mixture of L-glucose and L-mannose.

According to the process of the present invention, ions can be separated from a monosaccharide containing process stream by introducing the monosaccharide containing process stream onto one or more columns of a simulated moving bed chromatography apparatus and subsequently eluting the one or more columns to provide an extract stream that comprises the monosaccharide and a raffinate stream that comprises the ionic impurity or impurities. Preferably, the process is continuous, whereby the monosaccharide containing stream is continuously introduced into the apparatus while the one or more downstream fractions are continuously withdrawn. Additionally, the disclosed process can be part of a larger continuous process, which operates without interruption. Thus, in some aspects of the invention, the resins within the columns used in the simulated moving bed chromatography apparatus are not regenerated during the process. The larger industrial processes can therefore run without interruption that would otherwise be required to regenerate one or more columns in the simulated moving bed chromatography apparatus. Additionally, the disclosed processes allow for the simultaneous removal of all or substantially all (e.g., 70, 80, 85, 90, 95 or 99% by weight of) organic and/or inorganic cations and/or anions, thus addressing the need discussed above.

### Simulated Moving Bed Chromatography

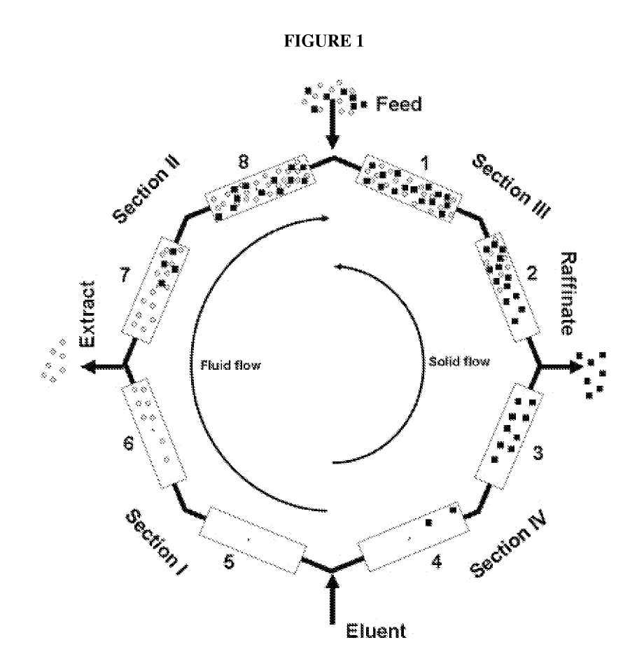

The process described herein uses simulated moving bed (SMB) chromatography to remove ions from saccharide mixtures (e.g., process streams that contain saccharides). Simulated moving bed chromatography is a technique that maintains the process features of continuous countercurrent flow chromatography without having to actually move the solid phase. Rather, a simulated movement of the solid phase is accomplished by continuously moving the various inlet and outlet ports of the chromatography unit in series throughout the chromatographic process. The simulated moving bed technique has been described in the literature, for example in R. A. Meyers, Handbook of Petroleum Refining Processes, pages 8-85 to 8-87, McGraw-Hill Book Company (1986), which is incorporated by reference herein for its teachings of SMB techniques. An illustration of a SMB process and apparatus is shown in FIG. 1.

Generally, solid packed columns are arranged in a ring formation made up of four sections with one or more columns per section (see FIG. 1). Two inlet streams (feed and eluent) and two outlets streams (extract and raffinate) are directed in alternating order to and from the column ring. Because the columns usually cannot be moved, the inlet and outlet position is switched at regular time intervals in the direction of the liquid flow, thus simulating countercurrent movement of columns.

The disclosed process is not limited to any particular type of simulated moving bed chromatography apparatus. Typically, however, a simulated moving bed chromatography apparatus comprises a plurality of columns connected together in a manner that allows each column to be eluted in either direction, depending on the elution phase cycle. The apparatus also typically comprises one or more conduits for charging eluent (desorbant), and one or more conduits for charging the mixture to be separated (feed) into the chromatography apparatus. The apparatus will also comprise one or more conduits for discharging liquid. Each of these conduits can be controlled by automatic valves, or by rotation of columns to the conduits. The number and size of the columns can be determined based on factors such as column-type, composition of the mixture, flow rate of the mixture, and concentration of the mixture.

One advantage of simulated bed chromatography is that the process can be carried out continuously, wherein various inlet and outlet streams are charged and withdrawn in a continuous manner, without interruption. Likewise, the position of the inlet and outlet streams can be changed relative to the series of columns, in equal shifts.

A variety of simulated moving bed apparatuses are available commercially. For example, a simulated moving bed apparatus suitable for use with the process disclosed herein is commercially available from Advanced Separation Technologies Incorporated, Lakeland, Fla. (Models LC1000 and ISEP LC2000), and Illinois Water Treatment (IWT), Rockford, Ill. (ADSEP system; see Morgart and Graaskamp, Paper No. 230, Continuous Process Scale Chromatography, The Pittsburgh Conference on Analytical Chemistry and Applied Spectroscopy, New Orleans, Feb. 22, 1988). Other suitable apparatuses with various configurations are specifically disclosed, for example, in U.S. Pat. Nos. 4,522,726 and 4,764,276, both of which are incorporated herein by this reference in their entirety for their teachings of simulated moving bed chromatography apparatuses.

In one embodiment, an ion exclusion resin is first contacted with the saccharide mixture (e.g., feed or process stream) and the resin is subsequently eluted with an aqueous eluent. During elution, there is a constant exchange of species between the stationary phase and the mobile phase, or the eluent (e.g., pure water). In one exemplary aspect, the anions chosen for separation as part of the overall process design are dianions, such as, for example, sulfate or phosphate. In ion exclusion chromatography, the more densely charged a species is, the more effectively it will be repelled from the inner surfaces of an ion exchange resin since those surfaces already contain a high concentration of charged residues. Given that some form of salt may be generated from the earlier steps of the process, it will be appreciated, that one advantage of the disclosed process is the choice of sulfate as a counterion from the standpoint of ease of separation from the monosaccharides by the SMB procedure from the monosaccharide mixtures.

### Ion Exclusion

An ion exclusion resin can be used to separate the ions from a saccharide mixture. In general, any ion exclusion resin can be used, for example, those that are either (e.g., strongly acidic sulfonated resins (a resin bearing sulfonic acid residues)) in their alkali metal form, or quaternary amine resins in a neutral form (chloride or sulfate as counterion). Typically the ion exclusion resin will comprise a cross-linked polymer to provide stability to the resin while also restricting the ability of the resin to swell. The ion exclusion resin is present in all columns used in the simulated moving bed chromatography apparatus. The ion exclusion resin is charged, and thus the raffinate resulting from the simulated moving bed chromatography tends to contain the ions, which move quickly through the column, while the nonionic species in the mixture, inter alia, the monosaccharides, are retained longer on the column and moves less quickly through the column. The ion exclusion resin can comprise the acid or anion form of the resin, depending on the specific process.

Ion exclusion systems may employ similar resins used in ion exchange systems, but differ in that the ionic functionality of the resin is the same as that of the electrolyte and, therefore, there is little to no net exchange of ions. In one aspect, the ion exclusion resin does not contain a mixture of strongly acidic resin (e.g., a resin bearing sulfonic acid residues) and weakly basic resin (e.g., a resin bearing tertiary amine groups); for instance, in one aspect, the ion exclusion resin does not include a “mixed bed.” In a further aspect, the ion exclusion resin can comprise a sulfonated polymer, for example, a sulfonated polystyrene with divinylbenzene (DVB) cross-linking which imparts physical stability to the resin polymer. The sulfonic acid functionality of the resin particles causes swelling in aqueous media. The resulting microporous resin particles can absorb water and nonionic solutes. The degree of molecular cross-linking with DVB influences the extent of sorption and prevents total dissolution of the porous resin. Because of ion repulsion and a high fixed acid chemical potential inside the resin microstructure, an electrolytic species, such as sulfuric acid in an acid/monosaccharide mixture, for example, is effectively prevented from entering the porous resin. However, the nonionic saccharides are free to diffuse into the resin structure. Thus, electrolytes will pass through a packed resin bed faster than nonelectrolytes which are held up or delayed within the resin's macroporous structure. In applying the disclosed process to effect an acid separation similar to the separation used in the acid exchange system, the resin used can be in its hydrogen form as opposed to the sodium form and, therefore, no ion exchange would occur in the system.

Specific examples of ionic exclusion resins that can be used with the methods described herein include DEAE SEPHADEX, QAE SEPHADEX, DEAE SEPHAROSE, DEAE-TRISACRYL PLUS, DEAE SEPHACEL, DEAE CELLULOSE, EXPRESS—ION EXCHANGER D, ECTEOLA CELLULOSE, PEI CELLULOSE, QAE CELLULOSE, EXPRESS ION EXCHANGER Q, which are available from Sigma-Aldrich Corporation, St. Louis, Mo., BIORAD AG-1X2, BIORAD AG-1X1, BIORAD AG-1X4, BIORAD AG-21K, BIORAD AG-1X8, BIORAD AG-1X0, BIORAD AG-2X4, BIORAD AG-2X8, BIORAD AG-2X10, BIOREX 9, AMBERLITE IRA-900, AMBERLITE IRA-938-C, AMBERLITE A-26, AMBERLITE IRA-400, AMBERLITE IRA-401S, AMBERLITE IRA-401, AMBERLITE IRA-400C, AMBERLITE IRP-67, AMBERLITE IRP-67M, AMBERLITE IRA-410, AMBERLITE IRA-910, DOWEX 1×2, DOWEX 1×4, DOWEX 21K, DOWEX MSA-1, DOWEX 1×8, DOWEX SBR, DOWEX 11, DOWEX MSA-2, DOWEX SAR, DOWEX 2×4, DUOLITE ES-11, DUOLITE A 101 D, IONAC A-540, IONAC A-544, IONAC A-548, IONAC A-546, IONAC A-550, IONAC A-5, IONAC A-580, IONAC A-590, IONAC AOOOO, QAE SEPHADEX A-25, QAE SEPHADEX A-50, DIAION TYPE I and DIAION TYPE II strong base anion exchangers. Strong base anion exchange resins include AMBERLITEIRP-67, BIORAD AG-1X10, BIORAD AG-1X8 and DOWEX 1X8. Another example is AMBERLITE IRP-67M. Yet another example is Purolite A600. Specific examples of anion exchange or exclusion silica-based chromatographic materials that may be used include Absorbosphere SAX, Baker Quaternary Amine, Bakerbond Quaternary Amine, Nucleosil SB, Partisil SAX, Progel-TSK DEAE-3SW, Progel-TSK DEAE-2SW, Sepherisorb S SAX, Supelcosil SAXI, Ultrasil-AX, and Zorbax SAX.

### Saccharide Mixtures

As discussed above, the disclosed process is directed to efficiently separating both inorganic and organic ionic by-products in one simultaneous operation from saccharide mixtures within process streams obtained in the synthesis of saccharides. A monosaccharide mixture can contain D- or L-monosaccharides. In one specific example, the monosaccharide mixture contains one or more L-monosaccharides. In a further specific example, the monosaccharide mixture contains L-mannose and L-glucose.

In general, any ions can be separated from saccharides using the disclosed process and thus the process is not limited to any particular type of ion. However, in some aspects, the ions can be ionic impurities resulting from monosaccharide synthesis. Such impurities can include, in various aspects, inorganic and organic acids and bases, and charged organic molecules. The exact nature of the ionic impurities will of course vary depending on the particular saccharide production process. Thus, the disclosed process can be applied to a variety of monosaccharide process streams that contain ions, which can be ionic impurities resulting from a monosaccharide synthesis. In other aspects, as discussed above, the saccharide mixture contains one or more dianions, such as sulfate or phosphate. In general, it will be appreciated that the disclosed process can be used to separate out all or substantially all ionic impurities present in a saccharide mixture without the need for multiple purification procedures, or even multiple chromatography passes. In one embodiment, the initial mixture has a conductivity of more than about 200, 400, 600, 800, 1000, 2000, or 4000 μSiemans/cm and the extract stream obtained by the process has a conductivity of less than about 750, less than about 500, less than about 300, less than about 250, less than about 200, less than about 150, less than about 100, less than about 50, or less than about 10 μSiemens/cm.

In a non-limiting exemplary aspect of the disclosed process, a mixture comprising L-mannose and L-glucose can be subjected to simulated moving bed chromatography as disclosed herein. Initially, the mixture comprises L-mannose and L-glucose and the following ionic impurities:

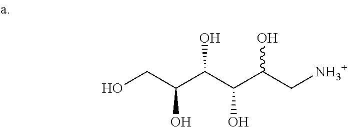 [SMILES: [NH3+]CC(O)[C@H](O)[C@@H](O)[C@@H](O)CO]

b. (3S,4S,5S)-2,3,4,5,6-pentahydroxyhexan-1-aminium
c. CH₃NH₃⁺ (methanaminium)
d. Na⁺ (sodium),
e. NH₄⁺ (ammonium); and
SO₄²⁻ (sulfate).

All of the ionic impurities, a-f, listed above, can be separated from the mixture in one continuous operation, thereby leaving an isolated aqueous fraction of L-mannose and L-glucose possessing low conductivity (<200 μSiemens/cm).

In certain aspects, the extract stream obtained by the disclosed processes can have a conductivity of less than about 1000 μSiemens/cm. In other aspects, the extract stream obtained by the process has a conductivity of less than about 750, less than about 500, less than about 300, less than about 250, less than about 200, less than about 150, less than about 100, less than about 50, or less than about 10 μSiemens/cm. In still another aspect, the extract stream obtained by the process has a conductivity of from about 1 to about 1000, from about 25 to about 800, from about 75 to about 600, or from about 100 to about 400 μSiemens/cm.

In one preferred embodiment L-mannose is removed or substantially removed from the mixture after SMB chromatography is performed.

While the ion exclusion resin within the SMB unit is continuously contacted with the mixture and eluted with water, a continuous stream containing the deionized monosaccharides is produced along with a second stream containing the ionic by-products.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is expressed in degrees C. or is at ambient temperature, and pressure is at or near atmospheric.

### Example

A solution of L-gluco- and L-mannocyanohydrins in 325 L of water was prepared by reacting 76 kg of L-arabinose with 50 kg sodium cyanide which had been almost completely neutralized by sulfuric acid, as described in U.S. Pat. No. 4,581,447, which is incorporated by reference herein in its entirety for its teachings of L-glucose synthesis. The resulting mixture of cyanohydrins was reduced in the presence of additional sulfuric acid using hydrogen and 5% palladium on carbon, and the resulting intermediate gluco and amino glycosides were hydrolyzed by adjusting the solution to pH 4-5 as described in U.S. Pat. No. 4,970,302, which is incorporated by reference herein in its entirety for its teachings of L-glucose synthesis. After removal of the hydrogenation catalyst by filtration, the resulting 717 kg aqueous solution was estimated to contain about 30 kg of L-glucose, about 55.7 kg of L-mannose, about 95.3 kg equivalent of sodium sulfate, and about 33.4 kg equivalent of ammonium sulfate. Also present were about 0.3 kg of L-mannonate ion, about 0.2 kg of L-gluconate ion (each in mixed sodium and ammonium forms), about 2.9 kg of the primary amine by-product derived from the over-reduction of mannosylamine, and about 1.6 kg of the primary amine by-product derived from the over-reduction of glucosylamine.

In order to reverse the proportion of L-glucose and L-mannose present, the above solution was diluted with an additional 950 kg of deionized water, treated with 2.3 kg of ammonium heptamolybdate, and then heated at 90° C. for almost 10 hours until a ratio of 68:32 of L-glucose to L-mannose was reached as determined by HPLC. The resulting solution was treated with activated carbon to reduce color, and filtered to provide 1,668 kg of feed solution for the ensuing deionization purification step.

The feed solution was maintained at 75° C. and was passed at a rate of 0.4 L per minute through a simulated moving-bed chromatography apparatus having 15 columns, each identically slurry-packed with 4 L of Dowex 99 (Sodium form) ion exchange resin and maintained at 65° C. The desorbant (deionized water) was also maintained at 75° C., and was passed into the simulated moving-bed system at a rate of 1.9 L per minute. Upon completion of the chromatographic separation, 4,452 kg of extract was obtained comprising only the purified monosaccharides in water as determined by NMR and conductivity measurements (<200 μSiemens/cm). The raffinate (16,288 kg) was confirmed to contain both the inorganic and organic ionic impurities as determined by conductivity and NMR measurements.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method of separating an ionic impurity from a monosaccharide-containing process stream, comprising:
a. contacting an ion exclusion resin within a simulated moving bed chromatography unit with the monosaccharide-containing process stream; and
b. eluting the ion exclusion resin with water to produce an extract stream that comprises monosaccharides and a raffinate stream that comprises the ionic impurity,
thereby separating the ionic impurity from the monosaccharide-containing process stream.

2. A method of separating an ionic impurity from a saccharide-containing process stream, comprising:
a. providing the saccharide containing process stream, wherein the process stream further comprises an inorganic dianion;
b. contacting an ion exclusion resin within a simulated moving bed chromatography unit with the saccharide-containing process stream; and
c. eluting the ion exclusion resin with an aqueous eluent to produce an extract stream that comprises saccharides and a raffinate stream comprises the ionic impurity,
thereby separating the ionic impurity from the saccharide-containing process stream.

3. The method of claim 1, wherein the method is continuous.

4. The method of claim 1, further comprising isolating the extract steam that comprises the monosaccharides or saccharides.

5. The method of claim 1, further comprising isolating the raffinate stream that comprises water soluble inorganic and organic salts of sodium and ammonium.

6. The method of claim 5, wherein the water soluble inorganic salts of sodium and ammonium comprise sodium sulfate and ammonium sulfate.

7. The method of claim 5, wherein the water soluble organic salts of sodium and ammonium comprise sodium aldonate and ammonium aldonates.

8. The method of claim 1, wherein the monosaccharide or saccharide containing process stream comprises an L-monosaccharide.

9. The method of claim 8, wherein the L-monosaccharide containing process stream comprises L-mannose and L-glucose.

10. The method of claim 1, wherein the ion exclusion resin is a cationic exclusion resin.

11. The method of claim 1, wherein the ion exclusion resin is an anionic exclusion resin.

12. The method of claim 1, wherein the ion exclusion resin comprises a cross-linked, sulfonated polymer.

13. The method of claim 1, wherein the ion exclusion resin comprises a cross-linked, sulfonated polymer is in its sodium salt form.

14. The method of claim 1, wherein the aqueous eluent is water.

15. The method of claim 1, wherein the method does not comprise adding a regenerant to the ion exclusion resin.

16. The method of claim 1, wherein the ionic impurity comprises both an organic and inorganic impurity.

17. The method of claim 2, wherein the dianion is a sulfate or phosphate ion.

18. The method of claim 1, wherein the extract stream has a conductivity of less than about 1000 μSeimens/cm.

19. The method of claim 1, wherein the extract stream has a conductivity of less than about 200 μSeimens/cm.

20. A continuous method of separating cationic and anionic impurities from a L-monosaccharide containing process stream, comprising:
a. contacting a sulfonated ion exclusion resin in its sodium salt form and within a simulated moving bed chromatography unit with the L-monosaccharide containing process stream; and
b. eluting the ion exclusion resin with water to produce an extract stream that comprises the L-monosaccharide and a raffinate stream that comprises the cationic and anionic impurities,
thereby separating the cationic and anionic impurities from the L-monosaccharide containing process stream.

21. L-glucose having a purity of at least 98%, wherein the L-glucose is at least substantially free of the following ionic impurities: 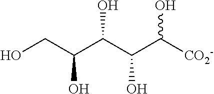
a. (3R,4S,5S)-2,3,4,5,6-pentahydroxyhexanoate 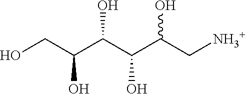 [SMILES: [NH3+]CC(O)[C@H](O)[C@@H](O)[C@@H](O)CO]
b. (3S,4S,5S)-2,3,4,5,6-pentahydroxyhexan-1-aminium
c. CH₃NH₃⁺ (methanaminium)
d. Na⁺ (sodium),
e. NH₄⁺ (ammonium); and
f. SO₄²⁻ (sulfate).

22. The L-glucose of claim 21, wherein the L-glucose has a purity of at least 99.5%.

23. The L-glucose of claim 21, wherein the L-glucose has a conductivity of less than about 200 μSiemens/cm.

24. L-glucose having a purity of at least 98%, wherein the L-glucose is at least substantially free of the following ionic impurities: 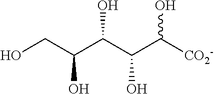
a. The monosodium salt of (3R,4S,5S)-2,3,4,5,6-pentahydroxyhexanoate 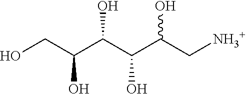 [SMILES: [NH3+]CC(O)[C@H](O)[C@@H](O)[C@@H](O)CO]
b. (3S,4S,5S)-2,3,4,5,6-pentahydroxyhexan-1-aminium
c. CH₃NH₃⁺ (methanaminium)
d. Na₂SO₄
e. (NH₄)₂SO₄; and
f. H₂Mo₇O₂₄⁻⁴.

25. The L-glucose of claim 24, wherein the L-glucose has a purity of at least 99.5%.

26. The L-glucose of claim 24, wherein the L-glucose has a conductivity of less than about 200 μSiemens/cm.

27. A pharmaceutical composition comprising the L-glucose of claim 21 and a pharmaceutically acceptable carrier or diluent.

28. A method for colonic cleansing comprising administering to a subject an effective amount of the L-glucose of claim 21.
